(19) United States
(12) Patent Application Publication
(10) Pub. No.: US 2008/0269266 A1
(43) Pub. Date: Oct. 30, 2008

(54) NOVEL COMPOUNDS 747

(75) Inventors: Thorsten Nowak, Macclesfield (GB); Andrew Peter Thomas, Macclesfield (GB); Stuart Charles Purkiss, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/055,658

(22) Filed: Mar. 26, 2008

Related U.S. Application Data
(60) Provisional application No. 60/908,223, filed on Mar. 27, 2007.

(51) Int. Cl. A61K 31/506; C07D 413/14; A61P 35/00

(52) U.S. Cl. 514/273; 544/296

(57) ABSTRACT

There is provided novel pyrimidine derivatives of formula (I)

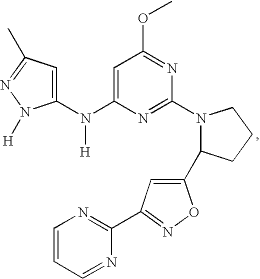

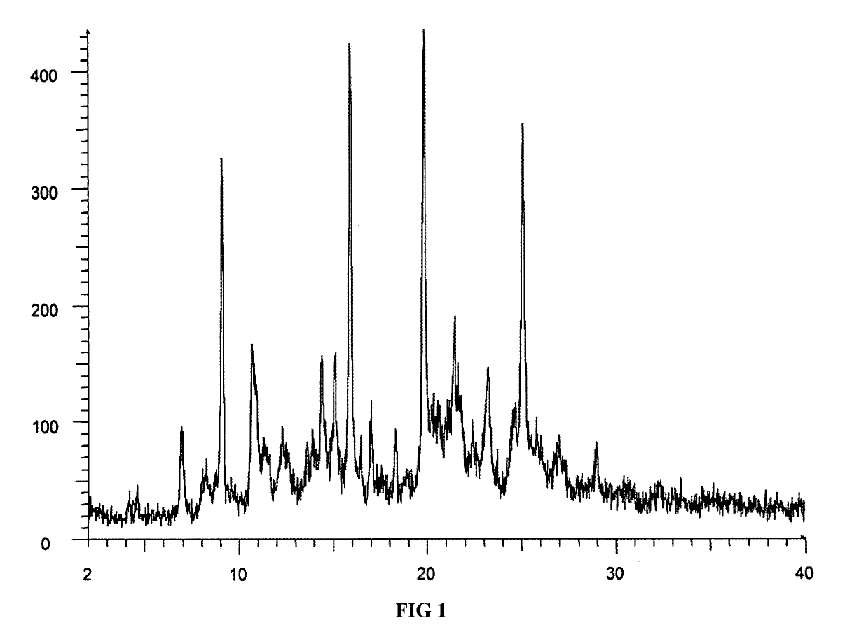

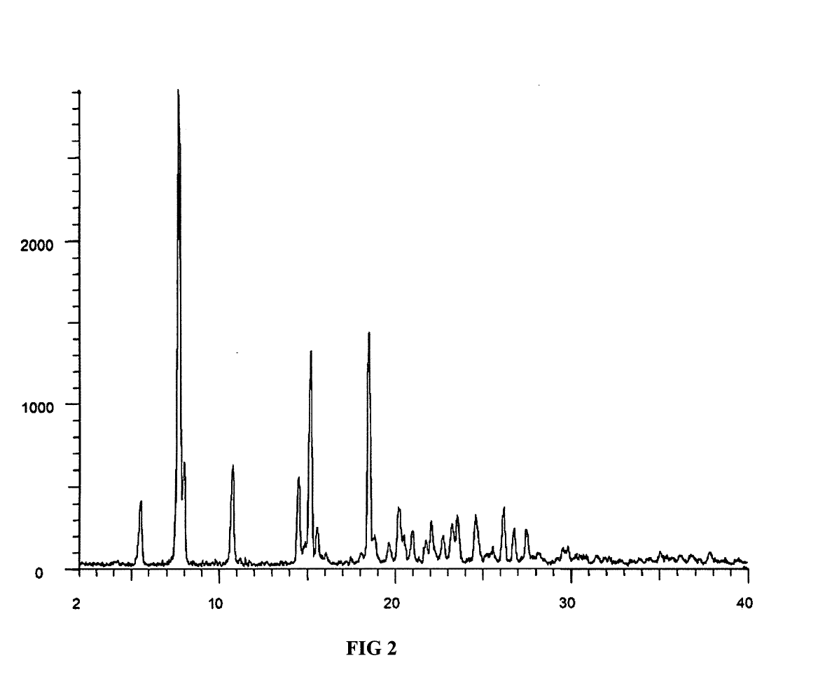

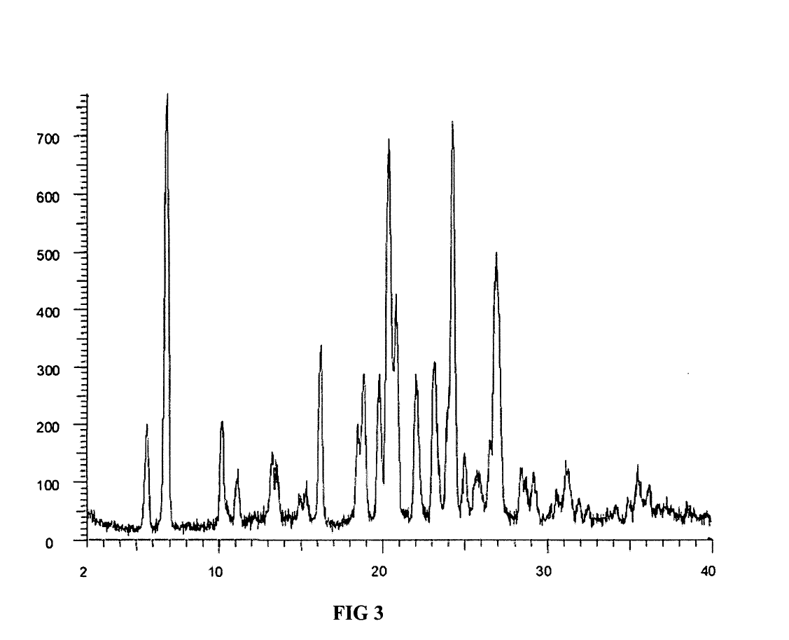

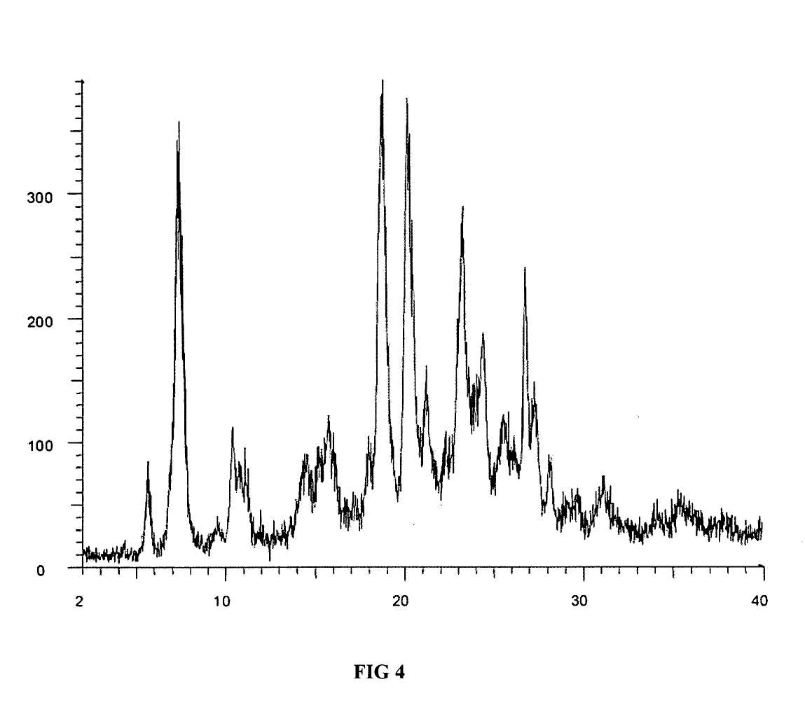

or pharmaceutically acceptable salts thereof, processes for their preparation, pharmaceutical compositions containing them and their use in therapy.

This application claims the benefit under 35 U.S.C. § 119(e) of U.S. Provisional Application No. 60/908,223 filed on Mar. 27, 2007.

The present invention relates to novel pyrimidine derivatives, processes for their preparation, pharmaceutical compositions containing them and their use in therapy.

The insulin-like growth factor (IGF) axis consists of ligands, receptors, binding proteins and proteases. The two ligands, IGF-I and IGF-II, are mitogenic peptides that signal through interaction with the type 1 insulin-like growth factor receptor (IGF-1R), a hetero-tetrameric cell surface receptor. Binding of either ligand stimulates activation of a tyrosine kinase domain in the intracellular region of the β-chain and results in phosphorylation of several tyrosine residues resulting in the recruitment and activation of various signalling molecules. The intracellular domain has been shown to transmit signals for mitogenesis, survival, transformation, and differentiation in cells. The structure and function of the IGF-1R has been reviewed by Adams et al (Cellular and Molecular Life Sciences, 57, 1050-1093, 2000). The IGF-IIR (also known as mannose 6-phosphate receptor) has no such kinase domain and does not signal mitogenesis but may act to regulate ligand availability at the cell surface, counteracting the effect of the IGF-1R. The IGF binding proteins (IGFBP) control availability of circulating IGF and release of IGF from these can be mediated by proteolytic cleavage. These other components of the IGF axis have been reviewed by Collett-Solberg and Cohen (Endocrine, 12, 121-136, 2000).

There is considerable evidence linking IGF signalling with cellular transformation and the onset and progression of tumours. IGF has been identified as the major survival factor that protects from oncogene induced cell death (Harrington et al, EMBO J, 13, 3286-3295, 1994). Cells lacking IGF-1R have been shown to be refractory to transformation by several different oncogenes (including SV40T antigen and ras) that efficiently transform corresponding wild-type cells (Sell et al., Mol. Cell Biol., 14, 3604-12, 1994). Upregulation of components of the IGF axis has been described in various tumour cell lines and tissues, particularly tumours of the breast (Surmacz, Journal of Mammary Gland Biology & Neoplasia, 5, 95-105, 2000), prostate (Djavan et al, World J. Urol., 19, 225-233, 2001, and O'Brien et al, Urology, 58, 1-7, 2001), lung (Liao et al, Chinese J of Cancer, 25, 1238-1242, 2006, and Minuto et al Cancer Res., 46, 985-988, 1986) and colon (Guo et al, Gastroenterology, 102, 1101-1108, 1992). Conversely, IGF-IIR has been implicated as a tumour suppressor and is deleted in some cancers (DaCosta et al, Journal of Mammary Gland Biology & Neoplasia, 5, 85-94, 2000). There is a growing number of epidemiological studies linking increased circulating IGF (or increased ratio of IGF-1 to IGFBP3) with cancer risk (Yu and Rohan, J. Natl. Cancer Inst., 92, 1472-1489, 2000). Transgenic mouse models also implicate IGF signalling in the onset of tumour cell proliferation (Lamm and Christofori, Cancer Res. 58, 801-807, 1998, Foster et al, Cancer Metas. Rev., 17, 317-324, 1998, and DiGiovanni et al, Proc. Natl. Acad. Sci., 97, 3455-3460, 2000).

Several in vitro and in vivo strategies have provided the proof of principal that inhibition of IGF-1R signalling reverses the transformed phenotype and inhibits tumour cell growth. These include neutralizing antibodies (Kalebic et al Cancer Res., 54, 5531-5534, 1994), antisense oligonucleotides (Resnicoff et al, Cancer Res., 54, 2218-2222, 1994), triple-helix forming oligonucleotides (Rinninsland et al, Proc. Natl. Acad. Sci., 94, 5854-5859, 1997), antisense mRNA (Nakamura et al, Cancer Res., 60, 760-765, 2000) and dominant negative receptors (D'Ambrosio et al., Cancer Res., 56, 4013-4020, 1996). Antisense oligonucleotides have shown that inhibition of IGF-1R expression results in induction of apoptosis in cells in vivo (Resnicoff et al, Cancer Res., 55, 2463-2469, 1995) and have been taken into man (Resnicoff et al, Proc. Amer. Assoc. Cancer Res., 40 Abs 4816, 1999). However, none of these approaches is particularly attractive for the treatment of major solid tumour disease.

Since increased IGF signalling is implicated in the growth and survival of tumour cells, and blocking IGF-1R function can reverse this, inhibition of the IGF-1R tyrosine kinase domain is an appropriate therapy by which to treat cancer. In vitro and in vivo studies with the use of dominant-negative IGF-1R variants support this. In particular, a point mutation in the ATP binding site which blocks receptor tyrosine kinase activity has proved effective in preventing tumour cell growth (Kulik et al, Mol. Cell Biol., 17, 1595-1606, 1997). Several pieces of evidence imply that normal cells are less susceptible to apoptosis caused by inhibition of IGF signalling, indicating that a therapeutic margin is possible with such treatment (Baserga, Trends Biotechnol., 14, 150-2, 1996).

There are few reports of selective IGF-1R tyrosine kinase inhibitors. Parrizas et al. described tyrphostins that had some efficacy in vitro and in vivo (Parrizas et al., Endocrinology, 138:1427-33 (1997)). These compounds were of modest potency and selectivity over the insulin receptor. Telik Inc. have described heteroaryl-aryl ureas which have selectivity over insulin receptors but potency against tumour cells in vitro is still modest (Published PCT Patent Application No. WO 00/35455).

Pyrimidine derivatives substituted at the 2- and 4-positions by a substituted amino group having IGF-1R tyrosine kinase inhibitory activity are described in WO 03/048133. Compounds in which the nitrogen atom of the amino substituent forms part of a heterocyclic ring are not disclosed.

WO 02/50065 discloses that certain pyrazolyl-amino substituted pyrimidine derivatives have protein kinase inhibitory activity, especially as inhibitors of Aurora-2 and glycogen synthase kinase-3 (GSK-3), and are useful for treating diseases such as cancer, diabetes and Alzheimer's disease. The compounds disclosed have a substituted amino substituent at the 2-position of the pyrimidine ring but again there is no disclosure of compounds in which the nitrogen atom of the amino substituent forms part of a heterocyclic ring.

Pyrazolyl-amino substituted pyrimidine derivatives having Aurora-2 and glycogen synthase kinase-3 (GSK-3) inhibitory activity in which the 2-position of the pyrimidine ring is substituted by an N-linked heterocyclic ring are disclosed generically in WO 02/22601, WO 02/22602, WO 02/22603, WO 02/22604, WO 02/22605, WO 02/22606, WO 02/22607 and WO 02/22608. In the large majority of the over four hundred compounds exemplified, the pyrimidine ring is present as part of a fused ring system, however, and in none of the exemplified compounds is the heterocyclic substituent at this position itself substituted by another ring substituent.

WO 01/60816 discloses that certain substituted pyrimidine derivatives have protein kinase inhibitory activity. There is no disclosure in WO 01/60816 of pyrimidine derivatives having a pyrazolyl-amino substituent at the 4-position on the pyrimidine ring and a substituted N-linked saturated monocyclic ring at the 2-position on the pyrimidine ring.

Pyrazolyl-amino substituted pyrimidine derivatives having IGF-IR tyrosine kinase inhibitory activity in which the 2-position of the pyrimidine ring is substituted by an N-linked heterocyclic ring are disclosed generically in WO2005/040159. There is no disclosure of the compound of Formula (I).

Whilst many of the compounds disclosed possess kinase activity and some may even display IGF-IR tyrosine kinase inhibitory activity, there is still a need for a compound which not only displays IGF-IR tyrosine kinase inhibitory activity but which possesses a balance of physical and biological properties.

In a first aspect of the present invention there is provided a compound of formula (I):

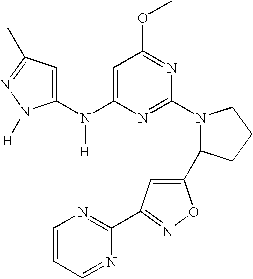

or a pharmaceutically acceptable salt thereof.
Figure A, X-Ray Powder Diffraction Pattern for (S)-6-Methoxy-2-{2-[3-(pyrimid-2-yl)isoxazol-5-yl]pyrrolidin-1-yl}-4-(5-methyl-1H-pyrazol-3-ylamino)pyrimidine Form 2.
Figure B, X-Ray Powder Diffraction Pattern for (S)-6-Methoxy-2-{2-[3-(pyrimid-2-yl)isoxazol-5-yl]pyrrolidin-1-yl}-4-(5-methyl-1H-pyrazol-3-ylamino)pyrimidine Form 1.
Figure C, X-Ray Powder Diffraction Pattern for (S)-6-Methoxy-2-{2-[3-(pyrimid-2-yl)isoxazol-5-yl]pyrrolidin-1-yl}-4-(5-methyl-1H-pyrazol-3-ylamino)pyrimidine Form 3.
Figure D, X-Ray Powder Diffraction Pattern for (S)-6-Methoxy-2-{2-[3-(pyrimid-2-yl)isoxazol-5-yl]pyrrolidin-1-yl}-4-(5-methyl-1H-pyrazol-3-ylamino)pyrimidine Form 4.

The compound of formula (I) is capable of existing in stereoisomeric forms. It will be understood that the invention encompasses all geometric and optical isomers of the compound of formula (I) and mixtures thereof including racemates. Tautomers and mixtures thereof also form an aspect of the present invention. It is to be understood that the compound of formula (I) above may exist in unsolvated forms as well as solvated forms, such as, for example, hydrated forms. Solvates and mixtures thereof also form an aspect of the present invention. For example, a suitable solvate of a compound of formula (I) is, for example, a hydrate such as a hemi-hydrate, a mono-hydrate, a di-hydrate or a tri-hydrate or an alternative quantity thereof. It is also to be understood the compound of the Formula I may exhibit polymorphism, and that the present invention encompasses all such forms which possess anticancer or antitumour activity. Thus, throughout the specification, where reference is made to the compound of formula (I), it is understood that the term compound includes isomers, mixtures of isomers, solvates, stereoisomers, and polymorphs that possess anticancer or antitumour activity.

The present invention relates to the compound of formula (I) as herein defined as well as to salts thereof. Salts for use in pharmaceutical compositions will be pharmaceutically acceptable salts, but other salts may be useful in the production of the compound of formula (I) and their pharmaceutically acceptable salts. Pharmaceutically acceptable salts of the invention may, for example, include acid addition salts of compound of formula (I), as herein defined, wherein the compound of formula (I) is sufficiently basic to form such salts, and base salts of compound of formula (I), as herein defined, wherein the compound of formula (I) is sufficiently acidic to form such salts. Such acid addition salts include but are not limited to fumarate, methanesulfonate, hydrochloride, hydrobromide, citrate and maleate salts and salts formed with phosphoric and sulfuric acid, and also salts formed by sulphonic acids such as ethane sulphonic acid, ethane disulphonic acid, benzene sulphonic acid and toluene sulphonic acid. Such base salts include but are not limited to alkali metal salts for example sodium salts, alkaline earth metal salts for example calcium or magnesium salts, and organic amine salts for example triethylamine, morpholine, N-methylpiperidine, N-ethylpiperidine, procaine, dibenzylamine, N,N-dibenzylethylamine or amino acids for example lysine. The compound of formula (I) is weakly basic and therefore may show a propensity to form salts with strong acids such as hydrochloric, hydrobromic, phosphoric, sulfuric acid, and sulphonic acids such as methane sulphonic acid, ethane sulphonic acid, ethane disulphonic acid, benzene sulphonic acid and toluene sulphonic acid.

The compounds of the formula (I) may also be administered in the form of a prodrug which is broken down in the human or animal body to give a compound of the formula (I). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

In a further aspect of the present invention there is provided a compound of formula (Ia):

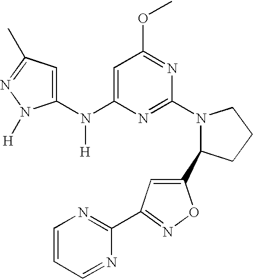

or a pharmaceutically acceptable salt thereof.

In a further aspect of the present invention there is provided a compound of formula (Ia) which is:
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 2, which has an X-ray powder diffraction pattern with one or more specific peaks at 2θ=6.906, 9.061, 10.693, 12.256, 14.393, 15.067, 15.903, 17.003, 18.317, 19.823, 21.458, 21.74, 20.603, 23.214, 24.635 and 25.061° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 1, which has an X-ray powder diffraction pattern with one or more specific peaks at 2θ=5.476, 7.671, 7.95, 10.749, 14.513, 15.172, 15.584, 18.507, 20.226, 20.983, 22.068, 23.251, 23.567, 24.607, 26.189, 26.796 and 27.512° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 3, which has an X-ray powder diffraction pattern with one or more specific peaks at 2θ=5.605, 6.808, 10.149, 13.221, 16.193, 18.506, 18.848, 19.814, 20.389, 20.827, 22.066, 23.159, 23.94, 24.288, 25.006, 26.54 and 26.924° when measured using CuKa radiation; or
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 4, which has an X-ray powder diffraction pattern with one or more specific peaks at 2θ=7.304, 7.56, 10.365, 15.766, 16.027, 17.982, 18.74, 20.172, 20.46, 21.234, 23, 23.249, 24.391, 25.516, 26.772 and 27.297° when measured using CuKa radiation.

In a further aspect of the present invention there is provided a compound of formula (Ia) which is:
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 2, which has an X-ray powder diffraction pattern with specific peaks at 2θ=9.061, 15.903, 19.823, and 25.061° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 1, which has an X-ray powder diffraction pattern with specific peaks at 2θ=7.671 and 18.507° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 3, which has an X-ray powder diffraction pattern with specific peaks at 2θ=6.808, 20.389, 20.827, 24.288 and 26.924° when measured using CuKa radiation; or
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 4, which has an X-ray powder diffraction pattern with specific peaks at 2θ=7.304, 7.56, 18.74, 20.172, 20.46, 23, 23.249, 24.391 and 26.772° when measured using CuKa radiation.

In a further aspect of the present invention there is provided a compound of formula (Ia) which is:
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 2, which has an X-ray powder diffraction pattern with specific peaks at 2θ=6.906, 9.061, 10.693, 12.256, 14.393, 15.067, 15.903, 17.003, 18.317, 19.823, 21.458, 21.74, 20.603, 23.214, 24.635 and 25.061° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 1, which has an X-ray powder diffraction pattern with specific peaks at 2θ=5.476, 7.671, 7.95, 10.749, 14.513, 15.172, 15.584, 18.507, 20.226, 20.983, 22.068, 23.251, 23.567, 24.607, 26.189, 26.796 and 27.512° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 3, which has an X-ray powder diffraction pattern with specific peaks at 2θ=5.605, 6.808, 10.149, 13.221, 16.193, 18.506, 18.848, 19.814, 20.389, 20.827, 22.066, 23.159, 23.94, 24.288, 25.006, 26.54 and 26.924° when measured using CuKa radiation; or
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 4, which has an X-ray powder diffraction pattern with specific peaks at 2θ=7.304, 7.56, 10.365, 15.766, 16.027, 17.982, 18.74, 20.172, 20.46, 21.234, 23, 23.249, 24.391, 25.516, 26.772 and 27.297° when measured using CuKa radiation.

Compounds of the present invention not only display IGF-IR tyrosine kinase inhibitory activity but also possess a balance of physical and biological properties. For example the compound of the present invention may ameliorate one or more properties such inhibition of Insulin Receptor, hERG, cytochrome P450 inhibition, LogD, solubility, protein binding, etc. Selective inhibition of Insulin-Like Growth Factor-1 Receptor over the inhibition of Insulin Receptor Phosphorylation may ameliorate effects on insulin signaling and the disruption of glucose homeostasis and associated toxicological effects. Differences in properties such as hERG or cytochrome P450 inhibition may result in an improved toxicological profile and may ameliorate drug:drug interactions. Differences in properties such as Log D, solubility or protein binding may result in lower drug metabolism, better absorption, and more drug available at the target site.

According to a further aspect of the present invention there is provided a process for the preparation of a compound of formula (I), as herein before described, comprising reacting a compound of formula (II):

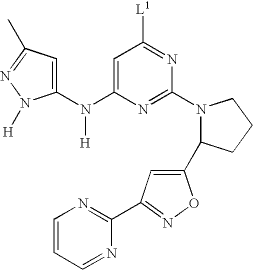

wherein L¹ is a leaving group (such as halogen, for example chlorine)
with a metal methoxide (such as an alkali metal or alkaline metal methoxide, for example sodium methoxide.

According to a further aspect of the present invention there is provided a process for the preparation of a compound of formula (I), as herein before described, comprising reacting a compound of formula (III):

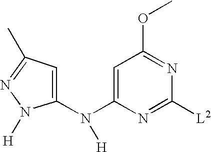

wherein L² is a leaving group (such as halogen, for example chlorine) with a compound of formula (IV)

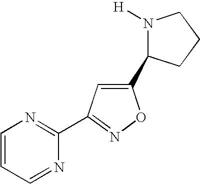

The reaction may take place in the presence of a metal salt, such zinc acetate.

According to a further aspect of the present invention there is provided a process for the preparation of a compound of formula (I), as herein before described, comprising

(i) reacting a compound of formula (V):

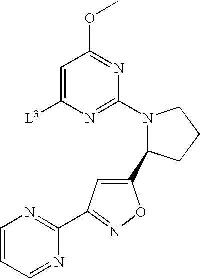

wherein L² is a leaving group (such as halogen, for example chlorine) with a compound of formula (VI)

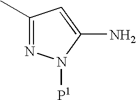

wherein P¹ is a protecting group (such as a BOC group), and

(ii) removing the protecting group P¹ to give a compound of formula (I).
Step (i) of this reaction may take place in the presence of a metal catalyst, such palladium acetate and 9,9-dimethyl-4,5-bis(diphenylphosphino)xanthene.

A compound of formula (II) may be prepared by reacting a compound of formula (VII)

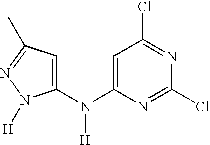

with a compound of formula (IV)

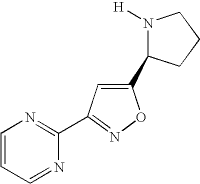

The reaction may take place in the presence of a metal salt, such zinc acetate.

A compound of formula (III) may be prepared by reacting a compound of formula (VIII)

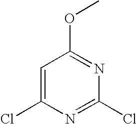

with a compound of formula (VI)

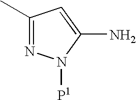

wherein P¹ is a protecting group (such as a BOC group), and

(ii) removing the protecting group P¹ to give a compound of formula (III).
Step (i) of this reaction may take place in the presence of a metal catalyst, such tris(dibenzylideneacetone)palladium and 9,9-dimethyl-4,5-bis(diphenylphosphino)xanthene.

A compound of formula (IV) may be prepared by

(i) reacting a compound of formula (IX)

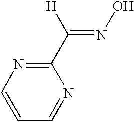

with a compound of formula (X)

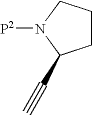

wherein P² is a protecting group (such as a BOC group), and

(ii) removing the protecting group P¹ to give a compound of formula (IV).

Alternatively, a compound of formula (IV) may be prepared by

(i) reacting a compound of formula (XI)

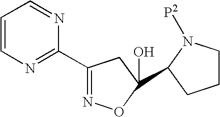

wherein P² is a protecting group (such as a BOC group),

with a dehydrating agent, and
(ii) removing the protecting group P¹ to give a compound of formula (IV).

Dehydrating agents include mixtures of SOCl₂ and tertiary amines such as triethylamine; mesyl chloride; and acetic anhydride.

A compound of formula (V) may be prepared by reacting a compound of formula (VIII)

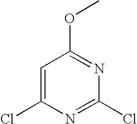

with a compound of formula (IV)

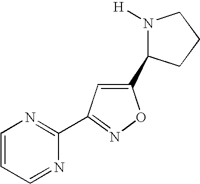

A compound of formula (VI) may be prepared by reacting 3-amino-5-methyl-1H-pyrazole with di-tert-butyl dicarbonate.

A compound of formula (VII) may be prepared by reacting 3-amino-5-methyl-1H-pyrazole with 2,4,6-trichloropyrimidine.

A compound of formula (VIII) may be prepared by reacting 2,4-dihydroxy-6-methoxypyrimidine with a halogenating agent, such as phosphorus (III) oxychloride.

A compound of formula (XI) may be prepared by

(i) reacting a compound of formula (XII)

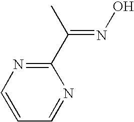

with a compound of formula (XIII)

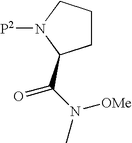

wherein P² is a protecting group (such as a BOC group) in the presence of a base (such as lithium di-isopropylamide).

A compound of formula (XI) may be prepared by

(i) reacting a compound of formula (XIV)

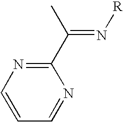

wherein R is a cycloalkyl group,

with a compound of formula (XV)

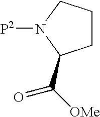

wherein P² is a protecting group (such as a BOC group) in the presence of a base (such as lithium di-isopropylamide).

A compound of formula (XII) may be prepared by reacting 2-cyanopyrimidine with methylmagnesium bromide to give the corresponding methylketone and reacting the methylketone with hydroxylamine.

A compound of formula (XIV) may be prepared by reacting 2-cyanopyrimidine with methylmagnesium bromide to give the corresponding methylketone and reacting the methylketone with an amine such as cyclohexylamine.

A compound of formula (XIII) may be prepared by reaction of 1-tert-butyl (2S)-pyrrolidine-1,2-dicarboxylate with N,O-dimethylhydroxylamine.

A compound of formula (XV) may be prepared by reaction of 1-tert-butyl (2S)-pyrrolidine-1,2-dicarboxylate with methanol.

2,4-dihydroxy-6-methoxypyrimidine may be prepared from barbaturic acid by reacting with a methylating agent such as methanol in the presence of boron trifluoride etherate.

It will be appreciated that the preparation of compounds of formula (I) may involve, at various stages, the addition and removal of one or more protecting groups. The protection and deprotection of functional groups is described in ‘Protective Groups in Organic Synthesis’, 2nd edition, T. W. Greene and P. G. M. Wuts, Wiley-Interscience (1991). For example, the BOC group may be removed using acid such as trifluoroactetic acid or hydrochloric acid.

When a pharmaceutically acceptable salt of a compound of formula (I) is required, for example an acid-addition salt, it may be obtained by, for example, reaction of said compound with a suitable acid using a conventional procedure.

As mentioned hereinbefore some of the compounds according to the present invention may contain one or more chiral centers and may therefore exist as stereoisomers. Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the compounds of formula (I), the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

The compound of formula (I) has activity as a pharmaceutical, in particular as a modulator or inhibitor of insulin-like growth factor-1 receptor (IGF-1R) activity, and may be used in the treatment of proliferative and hyperproliferative diseases/conditions, examples of which include the following cancers:

(1) carcinoma, including that of the bladder, brain, breast, colon, kidney, liver, lung, ovary, pancreas, prostate, stomach, cervix, colon, thyroid, esophagus and skin;
(2) hematopoietic tumours of lymphoid lineage, including acute lymphocytic leukaemia, B-cell lymphoma and Burketts lymphoma;
(3) hematopoietic tumors of myeloid lineage, including acute and chronic myelogenous leukaemias and promyelocytic leukaemia;
(4) tumors of mesenchymal origin, including fibrosarcoma and rhabdomyosarcoma; and
(5) other tumors, including melanoma, seminoma, tetratocarcinoma, neuroblastoma and glioma.

The compound of the invention are especially useful in the treatment of tumors of the breast, prostate, lung and colorectal area.

The compound of the invention are especially useful in the treatment of tumors of the breast and prostate.

According to a further aspect, therefore, the present invention provides a compound of formula (I), or a pharmaceutically acceptable salt thereof, as defined above for use in therapy of the human or animal body.

Thus, according to a further aspect of the present invention there is provided a compound of formula (I), or a pharmaceutically acceptable salt thereof, as defined above for use as a medicament.

In particular, the invention provides the use of a compound of formula (I), or a pharmaceutically acceptable salt thereof, as defined above in modulating insulin-like growth factor-1 receptor (IGF-1R) activity in a human or animal.

The invention also provides the use of a compound of formula (I), or a pharmaceutically acceptable salt thereof, as hereinbefore defined in the manufacture of a medicament for use in therapy, in particular in modulating insulin-like growth factor-1 receptor (IGF-1R) activity in a human or animal.

It will be appreciated that “therapy” also includes “prophylaxis” unless otherwise indicated. The terms “therapeutic” and “therapeutically” will be understood accordingly.

In a further aspect the present invention provides a method of treating cancer which comprises administering to a patient in need thereof a therapeutically effective amount of a compound of formula (I), or a pharmaceutically acceptable salt thereof, as hereinbefore defined.

In another aspect of the invention there is provided the use of a compound of formula (I), or a pharmaceutically acceptable salt thereof, as hereinbefore defined in the manufacture of a medicament for use in the treatment of cancer.

In another aspect of the invention there is provided a compound of formula (I), or a pharmaceutically acceptable salt thereof, as hereinbefore defined for use in the treatment of cancer.

The invention further provides a method of modulating insulin-like growth factor-1 receptor (IGF-1R) activity which comprises administering to a patient in need thereof a therapeutically effective amount of a compound of formula (I), or a pharmaceutically acceptable salt thereof, as hereinbefore defined.

In a further aspect the present invention provides a method of treating cancer which comprises administering to a patient in need thereof a therapeutically effective amount of a compound of formula (I), or a pharmaceutically acceptable salt thereof, as hereinbefore defined.

In a further aspect the present invention provides a method of treating cancer of the prostate, lung, colorectal area or breast which comprises administering to a patient in need thereof a therapeutically effective amount of a compound of formula (I), or a pharmaceutically acceptable salt thereof, as hereinbefore defined.

In a further aspect the present invention provides a method of treating cancer of the prostate or breast which comprises administering to a patient in need thereof a therapeutically effective amount of a compound of formula (I), or a pharmaceutically acceptable salt thereof, as hereinbefore defined.

In another aspect of the invention there is provided the use of a compound of formula (I), or a pharmaceutically acceptable salt thereof, as hereinbefore defined in the manufacture of a medicament for use in the treatment of cancer of the prostate, lung, colorectal area or breast.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, solutions, suspensions, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

It will be appreciated that the dosage administered will vary depending on the compound employed, the mode of administration, the treatment desired and the disorder indicated. Typically a daily dose of active ingredient in the range of from 0.5 mg to 75 mg active ingredient per kg body weight is received, given if required in divided doses, the precise amount of compound received and the route of administration depending on the weight, age, sex of the patient being treated and on the particular disease condition being treated according to principles known in the art.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of

5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a compound of formula (I), or a pharmaceutically acceptable salt thereof, as defined hereinbefore and an additional anti-tumour agent as defined hereinbefore for the conjoint treatment of cancer.

The activity and selectivity of compounds according to the invention may be determined using an appropriate assay as described, for example, in WO 03/048133, and as detailed below.

### EXAMPLES

The invention will now be further described with reference to the following illustrative examples. in which, unless stated otherwise:

(i) temperatures are given in degrees Celsius (° C.); operations were carried out at room or ambient temperature, that is, at a temperature in the range of 18 to 25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz, in DMSO-d₆ unless otherwise indicated. The following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms;
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺; and
(xi) the following abbreviations have been used:

| | |
|---|---|
| THF | tetrahydrofuran; |
| EtOAc | ethyl acetate; |
| DCM | dichloromethane; |
| DMSO | dimethylsulphoxide; |
| DIPEA | diisopropylethylamine; |
| NMP | N-methylpyrrolid-2-one; |
| tBuOH | tert-butyl alcohol; |
| TFA | trifluoroacetic acid; |
| DMF | N,N-dimethylformamide; and |
| DMA | N,N-dimethylacetamide. |

### XRPD (Analytical Instrument: Siemens D5000).

The X-ray powder diffraction spectra were determined by mounting a sample of the crystalline material on a Siemens single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+ software.

DSC (Analytical instrument: Mettler Toledo DSC820E)
DSC was recorded using a Mettler DSC820E with TSO801RO robotic system. Typically less than 5 mg of material, contained in a 40 μl aluminium pan fitted with a pierced lid, was heated over the temperature range 25° C. to 325° C. at a constant heating rate of 10° C. per minute. A nitrogen purge gas was used with flow rate 100 ml per minute.

### Example 1

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine

A mixture of (S)-6-chloro-2-{2-[3-(pyrimid-2-yl)isoxazol-5-yl]pyrrolidin-1-yl}-4-(5-methyl-1H-pyrazol-3-ylamino)pyrimidine (1.0 g, 2.36 mmol), and sodium methoxide (1.0 ml of a 28% solution in MeOH) in MeOH (10 ml) was heated at 120° C. in a sealed vessel under microwave irradiation for 6 hours. The reaction mixture was allowed to cool and evaporated under reduced pressure and the residue was treated with aqueous ammonium chloride solution and then extracted with DCM. The organic solution was washed with water and dried (Na₂SO₄) and the solvent removed by evaporation. The residue was purified by chromatography on silica (120 g column), eluting with EtOAc/hexanes (25:75 increasing in polarity to 100:0). The purified product was stirred and heated in EtOAc (20 ml) for 20 minutes. The solution was allowed to cool to ambient temperature and insoluble material removed by filtration. The solvent was removed from the filtrate by evaporation and the residue purified by chromatography on silica gel eluting with EtOAc. The purified product was dissolved in DCM (2 ml) and hexanes (approx. 50 ml) added. The precipitated solid was collected by filtration and dried under vacuum to give the title compound (0.32 g, 32%) as a white solid. NMR (DMSO-d₆@398K) 2.00-2.20 (m, 3H), 2.18 (s, 3H), 2.30-2.50 (m, 1H), 3.65-3.80 (m, 2H), 3.70 (s, 3H), 5.40-5.45 (d, 1H), 5.70-5.85 (br s, 1H), 5.85-6.00 (br s, 1H), 6.70 (s, 1H), 7.50-7.55 (t, 1H), 8.55-8.70 (br s, 1H), 8.90-8.95 (d, 2H), 11.35-11.50 (br s, 1H); m/z 420 [MH]+.

The starting materials were prepared by the following method:—

A mixture of 2,4,6-trichloropyrimidine (1.0 g, 5.4 mmol), 3-amino-5-methyl-1H-pyrazole (0.53 g, 5.4 mmol), and sodium carbonate (0.57 g, 5.4 mmol) in ethanol (25 ml) was stirred at ambient temperature for 18 hours. Water was added and the resulting precipitate was collected by filtration washed with water and a small amount of methanol, and dried to give 2,6-dichloro-4-(5-methyl-1H-pyrazol-3-ylamino)pyrimidine (1.15 g, 88%) as a colourless crystalline solid.

NMR (DMSO) 2.23 (s, 3H), 6.01 (s, 1H), 7.24 (s, 1H), 10.25 (br s, 1H), 11.9 (br s, 1H); m/z 244 [MH]+.

### (S)—N-tert-butoxycarbonyl-2-[3-(Pyrimid-2-yl)isoxazol-5-yl]pyrrolidine

13% Aqueous sodium hypochlorite solution (42.0 ml, 88.4 mmol) was slowly added to a mixture of (S)—N-tert-butoxycarbonyl-2-ethynlpyrrolidine (prepared as described in Bull. Soc. Chim. Fr. 1997, 134, 141-144 and J. Med. Chem. 1994, 37, 4455-4463) (11.2 g, 57.4 mmol) and pyrimidine-2-carbaldehyde oxime (4.80 g, 39.0 mmol, Khimiya Geterotsiklicheskikh Soedinenil (1972), 10, 1422-4) in DCM (250 ml) cooled in an ice bath maintaining the temperature of the mixture below 10° C. throughout the addition. The reaction mixture was then stirred for 18 hours at ambient temperature. The organic layer was separated off, and the aqueous layer was extracted with DCM. The combined organic extracts were washed with water, brine, dried (Na₂SO₄) and the solvent was removed by evaporation. The crude product was purified by chromatography on silica gel, eluting with EtOAc/Isohexane (50:50 increasing in polarity to 75:25) to give (S)—N-tert-butoxycarbonyl-2-[3-(pyrimid-2-yl)isoxazol-5-yl]pyrrolidine as a pale yellow oil, which crystallised. (2.30 g, 19%). NMR (DMSO-d₆): 1.78 (m, 3H), 2.14 (m, 1H), 2.92 (t, 2H), 4.36 (t, 1H), 6.82 (s, 1H), 7.60 (t, 1H), 8.96 (d, 2H); m/z 317 [MH]+.

(S)—N-tert-butoxycarbonyl-2-[3-(pyrimid-2-yl)isoxazol-5-yl]pyrrolidine (2.20 g, 6.96 mmol) was stirred in trifluoroacetic acid (50 ml) at ambient temperature for 18 hours. Excess trifluoroacetic acid was then removed by evaporation. The residue was basified to pH10 by addition of concentrated aqueous ammonia solution and extracted into ethyl acetate. The organic extracts were dried (Na₂SO₄) and the solvent was removed by evaporation. The residue was purified by column chromatography on silica gel eluting with methanol/ethyl acetate (5:95), then with methanol/dichloromethane (5:95) to give (S)-2-[3-(pyrimid-2-yl)isoxazol-5-yl]pyrrolidine (1.36 g, 91%) as a pale brown oil which later crystallised. NMR (CDCl₃) 1.85-2.12 (m, 3H), 2.20-2.34 (m, 1H), 3.10-3.26 (m, 2H), 4.50-4.75 (m, 3H), 6.85 (s, 1H), 7.26-7.32 (t, 1H), 8.78-8.82 (d, 2H); m/z 217 [MH]+.

A mixture of 2,6-dichloro-4-(5-methyl-1H-pyrazol-3-ylamino)pyrimidine (5.6 g, 23 mmol), (S)-2-[3-(pyrimid-2-yl)isoxazol-5-yl]pyrrolidine (5.19 g, 24 mmol) and zinc acetate (4.0 g, 25 mmol) was heated under reflux in isopropanol (250 ml) for 18 hours. The solution was cooled and the solvent removed by evaporation. Saturated aqueous ammonium chloride solution was added to the residue and the aqueous mixture was extracted with DCM (2×150 ml). The combined extracts washed with ammonium chloride solution, dried (MgSO₄) and the solvent removed by evaporation. The residue was purified by chromatography on silica gel eluting with EtOAc/Isohexane (50:100 increasing in polarity to 100:0). The purified product was triturated with ether and collected by filtration to give (S)-6-chloro-4-(5-methyl-1H-pyrazol-3-ylamino)-2-[2-{3-(pyrimid-2-yl)isoxazol-5-yl}pyrrolidin-1-yl]pyrimidine (5.2 g, 53%) as a white solid. NMR (DMSO-d₆ at 100° C.): 2.05-2.15 (m, 3H), 2.19 (s, 3H), 2.32-2.42 (m, 1H), 3.52-3.62 (m, 1H), 3.62-3.72 (m, 1H), 5.42 (d, 1H), 6.00 (s, 1H), 6.41 (s, 1H), 6.72 (s, 1H), 7.52 (dd, 1H), 8.90 (d, 2H), 9.21 (s, 1H), 11.62 (br s, 1H); m/z 424 [MH]+.

### Example 2

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine

2-Chloro-4-(1-tert-butoxycarbonyl-3-methylpyrazol-5-ylamino)-6-methoxypyrimidine (142 mg, 0.42 mmol) in isopropanol (3 ml) was heated at 150° C. in a sealed vessel under microwave irradiation for 10 minutes to give 2-chloro-6-methoxy-4-(5-methyl-1H-pyrazol-3-ylamino)pyrimidine in isopropanol. To this solution, (S)-2-[3-(pyrimid-2-yl)isoxazol-5-yl]pyrrolidine (100 mg, 0.46 mmol) zinc acetate (77 mg, 0.48 mmol) and isopropanol (2 ml) was added and the mixture heated under reflux for 17 hours. The solution was allowed to cool and the solvent removed by evaporation. Saturated aqueous ammonium chloride solution was added to the residue and the aqueous mixture was extracted with DCM (2×50 ml). The combined extracts were washed with water, dried (MgSO₄) and the solvent removed by evaporation. The residue was purified by chromatography on silica gel eluting with Isohexane/EtOAc (50:50). The purified product was triturated with DCM/Isohexane (10/90) to give the title compound (40 mg, 23%) as a solid.

NMR (DMSO-d₆ 398K) 2.00-2.20 (m, 3H), 2.18 (s, 3H), 2.30-2.50 (m, 1H), 3.65-3.80 (m, 2H), 3.70 (s, 3H), 5.40-5.45 (d, 1H), 5.70-5.85 (br s, 1H), 5.85-6.00 (br s, 1H), 6.70 (s, 1H), 7.50-7.55 (t, 1H), 8.55-8.70 (br s, 1H), 8.90-8.95 (d, 2H), 11.35-11.50 (br s, 1H); m/z 420 [MH]+.

The starting materials were prepared by the following method:—

Di-tert-butyl carbonate (24.0 g, 0.11 mol) in DCM (100 ml) was added to a stirred solution of 3-amino-5-methyl-1H-pyrazole (9.70 g, 0.1 mol) in a DCM (800 ml) and potassium hydroxide (188 ml of a 4.5M aqueous solution, 0.85 mol) at ambient temperature and the mixture stirred vigorously for 18 hours. The organic DCM solution was then separated, washed with water, brine and dried (MgSO₄). The solvent was removed by evaporation and the solid residue was recrystallised from EtOAc (50 ml) to give 5-amino-1-tert-butoxycarbonyl-3-methylpyrazole (4.6 g, 23%). NMR (DMSO-d₆) 1.54 (s, 3H), 2.00 (s, 3H), 5.15 (s, 1H), 6.20 (s, 2H).

A mixture of barbituric acid (19.5 g, 0.152 mol) and boron trifluoride etherate (75 ml) in methanol (300 ml) was heated and the ether removed by distillation. The mixture was then heated under reflux for 3 hours. The mixture was then cooled in an ice bath, solid material was collected by filtration and washed through with water. The solid was suspended in water heated to 100° C., allowed to cool and collected by filtration, washed with acetone/water and dried to give 2,4-dihydroxy-6-methoxypyrimidine (14.5 g, 67%). NMR 3.78 (3H, s), 4.93-4.94 (1H, m), 10.67 (1H, s), 11.26 (1H, s).

A mixture of 2,4-dihydroxy-6-methoxypyrimidine (15 g, 0.106 mol) in phosphorus (III) oxychloride (400 ml) was heated under reflux for 4 hours to give solution. Excess phosphorus (III) oxychloride was removed by evaporation, the residue treated with ice/water and extracted with EtOAc. The combined extracts were washed with water, dried (Na₂SO₄) and the solvent removed by evaporation to give 2,4-dichloro-6-methoxypyrimidine (5.5 g, 30%) as an oil. NMR_{—}3.96 (3H, s), 6.63 (1H, s); m/z_{—}179 [MH]⁺.

Tris(dibenzylideneacetone)palladium (0) (500 mg) and 9,9-dimethyl-4,5-bis(diphenylphosphino)xanthene (500 mg) were added to a stirred solution of 5-amino-1-tert-butoxycarbonyl-3-methylpyrazole (1.97 g, 10 mmol), 2,4-dichloro-6-methoxy-pyrimidine (1.80 g, 10 mmol) and cesium carbonate (5.20.g, 16 mmol) in dioxane (40 ml) under nitrogen. The mixture was heated at 82° C. for 18 hours, allowed to cool and insoluble material removed by filtration. The filter pad was washed with DCM and solvent was removed from the combined filtrate by evaporation. The residue was triturated with ether and the solid product purified by chromatography on silica gel eluting with EtOAc/Hexane (25:75 increasing in polarity to 50:50). The purified product was recrystallised from EtOAc/Hexane to give 2-Chloro-4-(1-tert-butoxycarbonyl-3-methylpyrazol-5-ylamino)-6-methoxypyrimidine (0.45 g, 13%) as a yellow solid.

NMR (DMSO-d₆) 1.52 (s, 9H), 2.20 (s, 3H), 3.87 (s, 3H), 6.41 (s, 1H), 6.48 (s, 1H), 9.84 (s, 1H).

### Example 3

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine

A mixture of (S)-4-chloro-6-methoxy-2-{2-[3-(pyrimid-2-yl)isoxazol-5-yl]pyrrolidin-1-yl}pyrimidine (756 mg, 2.0 mmol), 5-amino-1-tert-butoxycarbonyl-3-methylpyrazole (207 mg, 2.20 mmol) and cesium carbonate (1.0 g, 3.0 mmol) in dioxane (40 ml) was stirred and the solution purged with nitrogen for 15 minutes. Palladium (II) acetate (10 mg) and 9,9-dimethyl-4,5-bis(diphenylphosphino)xanthene (40 mg) were added and the solution was heated under reflux for 3 hours. Further palladium (II) acetate (10 mg) and 9,9-dimethyl-4,5-bis(diphenylphosphino)xanthene (40 mg) were added and the solution heated and stirred under reflux for 18 hours. The solution was allowed to cool, the insolubles were removed by filtration and the solvent removed form the filtrate by evaporation. The residue was dissolved in acetonitrile (10 ml) and the solution heated at 150° C. in a sealed vessel under microwave irradiation for 15 minutes. The solvent was removed by evaporation and the residue purified by chromatography on silica gel eluting with EtOAc/MeOH (100:0 increasing in polarity to 92:18) to give the title compound (140 mg, 17%).

NMR (DMSO-d₆@398K) 2.00-2.20 (m, 3H), 2.18 (s, 3H), 2.30-2.50 (m, 1H), 3.65-3.80 (m, 2H), 3.70 (s, 3H), 5.40-5.45 (d, 1H), 5.70-5.85 (br s, 1H), 5.85-6.00 (br s, 1H), 6.70 (s, 1H), 7.50-7.55 (t, 1H), 8.55-8.70 (br s, 1H), 8.90-8.95 (d, 2H), 11.35-11.50 (br s, 1H); m/z 420 [MH]+.

The starting material was prepared by the following method:—

A mixture of 2,4-dichloro-6-methoxy-pyrimidine (260 mg, 1.5 mmol), (S)-2-[3-(pyrimid-2-yl)isoxazol-5-yl]pyrrolidine (238 mg, 1.1 mmol) and zinc acetate (159 mg, 1.0 mmol) in isopropanol (4 ml) was heated under reflux for 18 hours. The reaction was allowed to cool and the solvent removed by evaporation. The residue was treated with aqueous ammonium chloride solution and the aqueous mixture extracted with DCM (×2). The organic extracts were washed with water and brine, and dried (Na₂SO₄) and the solvent removed by evaporation. The residue was purified by chromatography on silica gel eluting with EtOAc/Hexane (25:75) to give (S)-4-chloro-6-methoxy-2-{2-[3-(pyrimid-2-yl)isoxazol-5-yl]pyrrolidin-1-yl}pyrimidine (184 mg, 47%). NMR (DMSO-d₆ 373K) 2.05-2.22 (m, 3H), 2.33-2.50 (m, 1H), 3.58-3.85 (m, 2H), 3.82 (m, 3H), 5.36-5.50 (d, 1H), 6.13 (s, 1H), 6.77 (s, 1H), 7.53-7.55 (t, 1H), 8.90-8.91 (d, 2H); m/z 359 [MH]+.

### Example 4

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine—Form 2

20 mg of amorphous (S)-6-Methoxy-2-{2-[3-(pyrimid-2-yl)isoxazol-5-yl]pyrrolidin-1-yl}-4-(5-methyl-1H-pyrazol-3-ylamino)pyrimidine, was added to 1 ml of acetonitrile in a glass vial and warmed gently with agitation. The material dissolved and a further 20 mg (S)-6-Methoxy-2-{2-[3-(pyrimid-2-yl)isoxazol-5-yl]pyrrolidin-1-yl}-4-(5-methyl-1H-pyrazol-3-ylamino)pyrimidine was added and again gently warmed with agitation. This material again dissolved. The vial was set to one side and allowed to cool. After 30 minutes examination showed that there was a small amount of solid was present in the base of the vial. A further 10 mg of amorphous (S)-6-Methoxy-2-{2-[3-(pyrimid-2-yl)isoxazol-5-yl]pyrrolidin-1-yl}-4-(5-methyl-1H-pyrazol-3-ylamino)pyrimidine was added and the mixture was scratched vigorously with a stainless steel spatula. The mixture was allowed to stand without covering for 3 hrs. Visual examination showed that the vial had a layer of solid on the base which appeared to be crystalline. The solid was isolated and analysed by XRPD (see Figure A).

| Angle | Intensity | |
|---|---|---|
| 2-Theta ° | Count % | Intensity % |
| 19.823 | 437 | 100 |
| 15.903 | 425 | 97.3 |
| 25.061 | 355 | 81.2 |
| 9.061 | 326 | 74.6 |
| 21.458 | 190 | 43.5 |
| 10.693 | 161 | 36.8 |
| 14.393 | 157 | 35.9 |
| 15.067 | 157 | 35.9 |
| 23.214 | 146 | 33.4 |
| 21.74 | 120 | 27.5 |
| 20.603 | 117 | 26.8 |
| 17.003 | 117 | 26.8 |
| 24.635 | 111 | 25.4 |
| 6.906 | 95 | 21.7 |
| 12.256 | 95 | 21.7 |
| 18.317 | 93 | 21.3 |

DSC analysis of the isolated solid showed a broad endotherm from 25° C. to 80° C., which may indicate desolvation, and an endotherm with onset 127.7° C. and peak 134.8° C., corresponding to the melt of the material.

### Example 5

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine—Form 1

100 mg of amorphous (S)-6-Methoxy-2-{2-[3-(pyrimid-2-yl)isoxazol-5-yl]pyrrolidin-1-yl}-4-(5-methyl-1H-pyrazol-3-ylamino)pyrimidine was placed in a glass vial. A Teflon coated magnetic stirrer flea was added then sufficient acetonitrile to cover the sample was added. The vial was sealed and the mixture was stirred at room temperature for 3 days. The solid was filtered off and dried by suction. The dry solid was analysed by XRPD (see Figure B)

| Angle | Intensity | |
|---|---|---|
| 2-Theta ° | Count % | Intensity % |
| 7.671 | 2917 | 100 |
| 18.507 | 1435 | 49.2 |
| 15.172 | 1323 | 45.4 |
| 7.95 | 644 | 22.1 |
| 10.749 | 618 | 21.2 |
| 14.513 | 551 | 18.9 |
| 5.476 | 412 | 14.1 |
| 20.226 | 356 | 12.2 |
| 24.607 | 328 | 11.2 |
| 26.189 | 322 | 11 |
| 23.567 | 322 | 11 |
| 22.068 | 286 | 9.8 |
| 23.251 | 269 | 9.2 |
| 15.584 | 247 | 8.5 |
| 26.796 | 242 | 8.3 |
| 27.512 | 233 | 8 |
| 20.983 | 228 | 7.8 |

DSC analysis showed no significant events below 100° C. and an endotherm with onset 136.0° C. and peak 147.1° C., corresponding to the melting of the material.

The experiment was repeated with 250 mg of amorphous (S)-6-Methoxy-2-{2-[3-(pyrimid-2-yl)isoxazol-5-yl]pyrrolidin-1-yl}-4-(5-methyl-1H-pyrazol-3-ylamino)pyrimidine, and gave similar results.

### Example 6

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine—Form 3

20 mg of the Form 2 (S)-6-Methoxy-2-{2-[3-(pyrimid-2-yl)isoxazol-5-yl]pyrrolidin-1-yl}-4-(5-methyl-1H-pyrazol-3-ylamino)pyrimidine prepared by the method of Example 4 was placed in a glass vial with a magnetic stirrer flea. Sufficient Water containing 5% Methanol was added and the mixture was stirred at room temperature for 3 days. The solid was filtered off and dried briefly by suction. The solid was analysed by XRPD (see Figure C).

| Angle | Intensity | |
|---|---|---|
| 2-Theta ° | Count % | Intensity % |
| 6.808 | 751 | 100 |
| 24.288 | 725 | 96.5 |
| 20.389 | 694 | 92.4 |
| 26.924 | 499 | 66.4 |
| 20.827 | 427 | 56.9 |
| 16.193 | 325 | 43.3 |
| 23.159 | 308 | 41 |
| 18.848 | 287 | 38.2 |
| 22.066 | 286 | 38.1 |
| 19.814 | 286 | 38.1 |
| 23.94 | 222 | 29.6 |
| 10.149 | 202 | 26.9 |
| 18.506 | 199 | 26.5 |
| 5.605 | 198 | 26.4 |
| 26.54 | 171 | 22.8 |
| 13.221 | 151 | 20.1 |
| 25.006 | 147 | 19.6 |

DSC analysis showed a broad endotherm from ambient to 85° C., which may indicate dehydration of a hydrate, and a broad endotherm onset 120.4° C. peak 134.0° C., corresponding to the melt of the non-solvent containing material.

### Example 7

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine—Form 3

20 mg of the Form 1 (S)-6-Methoxy-2-{2-[3-(pyrimid-2-yl)isoxazol-5-yl]pyrrolidin-1-yl}-4-(5-methyl-1H-pyrazol-3-ylamino)pyrimidine prepared by the method of Example 5 was placed in a glass vial with a magnetic stirrer flea. Sufficient Water containing 5% Methanol was added and the mixture was stirred at room temperature for 3 days. The solid was filtered off and dried briefly by suction. The solid was analysed by XRPD and DSC and shown to be the same form as isolated from example 6.

### Example 8

### Preparation of (S)-6-Methoxy-2-{2-[3-(pyrimid-2-yl)isoxazol-5-yl]pyrrolidin-1-yl}-4-(5-methyl-1H-pyrazol-3-ylamino)pyrimidine—Form 4

20 mg of the Form 1 (S)-6-Methoxy-2-{2-[3-(pyrimid-2-yl)isoxazol-5-yl]pyrrolidin-1-yl}-4-(5-methyl-1H-pyrazol-3-ylamino)pyrimidine prepared by the method of Example 5 was placed in a glass vial with a magnetic stirrer flea. Sufficient isopropanol to cover the sample was added and the mixture was stirred at room temperature for 3 days. The solid was filtered off and dried briefly by suction. The solid was analysed by XRPD (see Figure D).

| Angle | Intensity | |
|---|---|---|
| 2-Theta ° | Count % | Intensity % |
| 18.74 | 380 | 100 |
| 20.172 | 377 | 99.2 |
| 7.304 | 334 | 87.9 |
| 23.249 | 290 | 76.3 |
| 7.56 | 267 | 70.3 |
| 26.772 | 241 | 63.4 |
| 20.46 | 235 | 61.8 |
| 23 | 197 | 51.8 |
| 24.391 | 187 | 49.2 |
| 21.234 | 161 | 42.4 |
| 27.297 | 148 | 38.9 |
| 25.516 | 122 | 32.1 |
| 15.766 | 121 | 31.8 |
| 10.365 | 112 | 29.5 |
| 16.027 | 107 | 28.2 |
| 17.982 | 104 | 27.4 |

DSC analysis showed no significant events below 100° C., an endotherm with onset 128.0° C. and peak 138.4° C., melt. of material.

### Intermediate 1: Preparation of (S)-tert-butyl 2-[3-(pyrimid-2-yl)isoxazol-5-yl]pyrrolidine-1-carboxylate

Triethylamine (63.8 ml, 457.85 mmol) and 2-methyltetrahydrofuran (200 ml) were added to the organic phase (S)-tert-butyl 2-((S)-5-hydroxy-3-(pyrimidin-2-yl)-4,5-dihydroisoxazol-5-yl)pyrrolidine-1-carboxylate (21.87 g, 65.41 mmol) and cooled to −20° C. A solution of thionyl chloride (14.31 ml, 196.22 mmol) in 2-methyltetrahydrofuran (100 ml) was added drop wise to the reaction, keeping the internal temperature below 0° C. The reaction was left stirring for 3 hours at 0° C. The reaction was monitored by LCMS and TLC (10% methanol/ethylacetate). Dichloromethane (1.0 litre) and water (440 ml) were then added. The organic layer was separated and treated with 2M aqueous sodium hydroxide solution (400 ml), stirred for 10 minutes. The aqueous layer was extracted with dichloromethane (250 ml). The organics were combined and washed with water (1.0 litre), brine (1.0 litre), dried (magnesium sulphate), filtered and the solvent removed in vacuo. Gave 20.0 g of a crude brown oil. This crude product was purified by flash silica chromatography, eluting with 100% ethylacetate. Pure fractions were evaporated to dryness to afford 8.0 g of (S)-tert-butyl 2-(3-(pyrimidin-2-yl)isoxazol-5-yl)pyrrolidine-1-carboxylate (39%) as a brown oil.

¹H NMR (400.132 MHz, DMSO) δ 1.26 (9H, s), 1.91-2.06 (3H, m), 2.23-2.38 (1H, m), 3.28-3.45 (1H, m), 3.50-3.57 (1H, m), 5.00-5.11 (1H, m), 6.87 (1H, s), 7.64 (1H, t), 9.00 (2H, d); m/z (M+H)+, 261

Enantiomeric excess=78% by chiral HPLC (Chiralpak AD 5 micron column; mobile phase iso-hexane/isopropyl alcohol/triethylamine 80:20:0.1). The material was purified by preparative chiral HPLC (20 cm column packed with 20 micron Chiralpak AD; eluant=isohexane/ethanol/methanol, 95:2.5:2.5; 50 g loadings; 60 min runtime) to give the desired enantiomer as a yellow solid (6.5 g) with an e.e. of 99.2%.

(S)-tert-butyl 2-((S)-5-hydroxy-3-(pyrimidin-2-yl)-4,5-dihydroisoxazol-5-yl)pyrrolidine-1-carboxylate may be prepared as follows:

### (S)-tert-butyl 2-((S)-5-hydroxy-3-(pyrimidin-2-yl)-4,5-dihydroisoxazol-5-yl)pyrrolidine-1-carboxylate

n-Butyllithium (1.6M solution in hexanes) (102 ml, 163.56 mmol) was added drop wise over 15 minutes to a solution of Diisopropylamine (22.92 ml, 163.56 mmol) in 2-methyltetrahydrofuran (68 ml) at −10° C., under a nitrogen atmosphere. The reaction was stirred for 10 minutes before the drop wise addition over 20 minutes of a thick brown solution of N-(1-(pyrimidin-2-yl)ethylidene)cyclohexanamine (33.2 g, 163.56 mmol) in 2-methyltetrahydrofuran (30 ml). The reaction was stirred for a further 30 minutes. A solution of (S)-1-tert-butyl 2-methylpyrrolidine-1,2-dicarboxylate (15.0 g, 65.42 mmol) in 2-methyltetrahydrofuran (60 ml) was then added over 5 minutes. The reaction was stirred for 30 minutes and then warmed to 25° C. and for 3 hours. The reaction was monitored by LCMS & TLC (10% methanol/ethylacetate). 10% ammonium chloride aqueous solution (150 ml) was then added carefully. The layers were separated and the organic layer was used in the next step. m/z (M+H)+, 401

The organic phase (S,Z)-tert-butyl 2-(3-(cyclohexylamino)-3-(pyrimidin-2-yl)acryloyl)pyrrolidine-1-carboxylate (26.2 g, 65.42 mmol) in 2-methyltetrahydrofuran (260 ml) was added to Hydroxylamine hydrochloride (6.81 ml, 163.54 mmol). The reaction was heated to reflux and stirred for 4 hours. LCMS & TLC (10% methanol/ethylacetate) indicated product formation. The reaction was cooled to room temperature and filtered. The filter cake was washed with 2-methyltetrahydrofuran (2×50 ml). Dichloromethane (1.0 litre) was added to the filtrate, this was washed with water (1.0 litre), 50% brine/water (1.0 litre), dried (magnesium sulphate) and filtered. The organic solution was used in the preparation of (S)-tert-butyl 2-[3-(pyrimid-2-yl)isoxazol-5-yl]pyrrolidine-1-carboxylate. m/z (M−H)−, 333.

N-(1-(pyrimidin-2-yl)ethylidene)cyclohexanamine may be prepared as follows:

Cyclohexylamine (37.5 ml, 327.53 mmol) was added to a stirred mixture of 1-(pyrimidin-2-yl)ethanone (20 g, 163.77 mmol) in toluene (60.0 ml). The reaction was heated at reflux employing a Dean-Stark trap to remove water. After 3 hours the reaction was judged complete by GCMS. The brown solution was cooled and concentrated in vacuo. Gave 35 g of N-(1-(pyrimidin-2-yl)ethylidene)cyclohexanamine a crude brown oil. The product was used immediately in the reaction above.

1-(pyrimidin-2-yl)ethenone may be prepared as follows:

A solution of pyrimidine-2-carbonitrile (75 g, 713.62 mmol) in THF (750 ml, 10 vol) was added dropwise to a solution of Methylmagnesium bromide (3.0M in diethylether) (357 ml, 1070.44 mmol) in THF (750 ml) at −5° C. The resulting yellow suspension/solution was stirred at 0° C. overnight, and added to a rapidly stirred mixture of saturated ammonium chloride solution (750 ml) and 4M HCl (450 ml) at 5° C., then pH adjusted to 1 with additional 2M HCl (5 mL). The solution was warmed to 20° C., stirred for 40 minutes, cooled to 0° C. then pH adjusted to 6.5-7 by addition of saturated K₂CO₃ solution (37.5 ml), warmed to 10° C. and separated. The aqueous phase was further extracted into ethyl acetate (5×750 ml). Sodium chloride was added to saturate the aqueous phase, which was extracted further into ethyl acetate (750 ml). The pH of the aqueous phase was adjusted to 7-8 by addition of saturated K₂CO₃ solution, and extracted further with ethyl acetate (3×750 ml). The combined organics were washed with saturated brine (750 ml), dried over MgSO₄, filtered and concentrated in vacuo to give 78.9 g of a brown solid. The crude product was purified by flash silica chromatography, elution in EtOAc. Pure fractions were evaporated to dryness to afford 1-(pyrimidin-2-yl)ethanone (65.0 g, 74.6%) as a yellow crystalline solid.

¹H NMR (400.132 MHz, DMSO) δ 2.67 (3H, s), 7.72 (1H, t), 9.02 (2H, d); m/z (M+H)+, 123.

(S)-1-tert-butyl 2-methylpyrrolidine-1,2-dicarboxylate may be prepared as follows:

N,N′-Carbonyldiimidazole (67.8 g, 418.13 mmol) and 2-methyltetrahydrofuran (375 ml) were charged to a 3 litre vessel. The slurry was allowed to stir at 25° C. for 10 minutes. A solution of (S)-1-(tert-butoxycarbonyl)pyrrolidine-2-carboxylic acid (75 g, 348.44 mmol) in 2-methyltetrahydrofuran (375 ml) was then added drop wise over 10 minutes. The white slurry turned into a clear pale yellow solution. The reaction was stirred for 2 hours at 25° C. Methyl alcohol (70.6 ml, 1742.19 mmol) was then added. The reaction mixture was then stirred at reflux for 2 hours. The reaction was cooled to 25° C. and left to stir for 1 hour. The reaction mixture was then washed with 10% w/v citric acid (2×375 ml), dried (magnesium sulphate), filtered and the solvent removed in vacuo. Gave 89 g crude. The crude was dissolved in 50% ethylacetate/isohexane (180 ml) and passed through a silica pad eluting with 50% ethylacetate/isohexane (3.0 litres). The filtrate was evaporated to dryness to afford 70 g of (S)-1-tert-butyl 2-methylpyrrolidine-1,2-dicarboxylate a clear oil (88%).

¹H NMR (400.132 MHz, DMSO) δ 1.27-1.45 (9H, m), 1.75-1.91 (3H, m), 2.08-2.29 (1H, m), 3.21-3.42 (2H, m), 3.56-3.69 (3H, m), 4.09-4.23 (1H, m); m/z (M−H)−, 228

### Intermediate 1: Alternate preparation of (S)-tert-butyl 2-[3-(pyrimid-2-yl)isoxazol-5-yl]pyrrolidine-1-carboxylate

Triethylamine (7.88 ml, 56.52 mmol) and 2-methyltetrahydrofuran (62 ml) were added to (S)-tert-butyl 2-((S)-5-hydroxy-3-(pyrimidin-2-yl)-4,5-dihydroisoxazol-5-yl)pyrrolidine-1-carboxylate (2.7 g, 8.07 mmol) and the reaction was cooled to −20° C. A solution of thionyl chloride (1.767 ml, 24.22 mmol) in 2-methyltetrahydrofuran (12 ml) and added drop wise to the reaction, keeping the internal temperature below 0° C. The reaction was left stirring for 3 hours at 0° C. The reaction looked complete by LCMS and TLC (10% methanol/ethylacetate). Dichloromethane (100 ml) and water (50 ml) were then added. The organic layer was separated and treated with 2M aqueous sodium hydroxide solution (50 ml), stirred for 10 minutes. The aqueous layer was extracted with dichloromethane (30 ml). The organics were combined and washed with water (100 ml), brine (100 ml), dried (magnesium sulphate), filtered and the solvent removed in vacuo. Gave 3.6 g of a crude brown oil. This crude product was purified by flash silica chromatography, eluting with 100% ethylacetate. Pure fractions were evaporated to dryness to afford 2.1 g of (S)-tert-butyl 2-(3-(pyrimidin-2-yl)isoxazol-5-yl)pyrrolidine-1-carboxylate (82%) as a crystalline white solid.

¹H NMR (400.132 MHz, DMSO) δ 1.26 (9H, s), 1.91-2.06 (3H, m), 2.23-2.38 (1H, m), 3.28-3.45 (1H, m), 3.50-3.57 (1H, m), 5.00-5.11 (1H, m), 6.87 (1H, s), 7.64 (1H, t), 9.00 (2H, d).

(S)-tert-butyl 2-((S)-5-hydroxy-3-(pyrimidin-2-yl)-4,5-dihydroisoxazol-5-yl)pyrrolidine-1-carboxylate may be prepared as follows:

n-Butyllithium (120 ml, 191.63 mmol) was charged to a 3 litre reactor. Tetrahydrofuran (75 ml) was added and the mixture cooled to −40° C. Diisopropylamine (26.9 ml, 191.63 mmol) was then added drop-wise over 20 minutes, left stirring at −40° C. for 30 minutes. A slurry of 1-(pyrimidin-2-yl)ethanone oxime (13.14 g, 95.81 mmol) in tetrahydrofuran (75 ml) was then added portion wise over 30 minutes. The reaction was left stirring at −40° C. for 30 minutes before warming to 0° C. and left to stir for 2 hours. The reaction was then cooled to −5° C. and a solution of (S)-tert-butyl 2-(methoxy(methyl)carbamoyl)pyrrolidine-1-carboxylate (7.5 g, 29.03 mmol) in tetrahydrofuran (15 ml) was added drop wise over 10 minutes. The reaction was left stirring at 0° C. overnight. Water (7.5 ml) was added to the reaction dropwise over 5 minutes. The mixture was then partitioned between water (150 ml) and ethylacetate (150 ml). The organic layer was separated and washed with water (75 ml), saturated aqueous citric acid solution (2×140 ml), water (75 ml), brine (75 ml), dried (magnesium sulphate), filtered and the solvent removed in vacuo. Gave 12 g crude. This crude product was purified by flash silica chromatography, eluting with 10% methanol/ethylacetate. Pure fractions were evaporated to dryness to afford 2.7 g of (S)-tert-butyl 2-((S)-5-hydroxy-3-(pyrimidin-2-yl)-4,5-dihydroisoxazol-5-yl)pyrrolidine-1-carboxylate (28%) as a yellow oil.

(M−H)− 333

tert-Butyl (2S)-2-(methoxy-methylcarbamoyl)pyrrolidine-1-carboxylate may be prepared as follows:

Dimethylaminopyridine (4.0 Kg, 32.7 mol) was added over 15 minutes to a mixture of (2S)-1-[(2-methylpropan-2-yl)oxycarbonyl]pyrrolidine-2-carboxylic acid (2.0 Kg, 9.3 mol), N-(3-dimethylaminopropyl)-N′-ethylcarbodiimide hydrochloride (2.0 Kg, 10.4 mol) and N,O-dimethylhydroxylamine.hydrochloride (2.6 Kg, 26.7 mol) in dichloromethane (30.0 litres) at 25° C. The reaction was left stirring at 25° C. for 48 hrs (during this time a white precipitate came out of solution). The reaction monitored by HPLC (210 nm), TLC: 50% ethylacetate/isohexane, stain: PMA. The reaction was transferred to a separator with dichloromethane (5.0 litres) & water (15.0 litres). The aqueous layer was separated and extracted with dichloromethane (10.0 litres). The organics were combined and washed with water (15.0 litres), dried (magnesium sulphate), filtered and the solvent removed in vacuo. Gave an oil/solid. 50% Ethylacetate/isohexane (10.0 litres) was added, the solid was filtered off and washed with 40% ethylacetate/isohexane (2.0 litres) before being disposed off. The solvent was removed in vacuo from the filtrate. This crude product was purified by flash silica chromatography, eluting with 50% ethylacetate/isohexane. Pure fractions were evaporated to dryness and azeotroped with toluene (2×5.0 litres) to afford 1.72 Kg of tert-butyl (2S)-2-(methoxy-methylcarbamoyl)pyrrolidine-1-carboxylate (72%) as a clear oil.

¹H NMR (400.132 MHz, CDCl3) δ 1.33-1.55 (9H, m), 1.75-2.09 (3H, m), 2.09-2.31 (1H, m), 3.20 (3H, s), 3.33-3.66 (2H, m), 3.76 (3H, d), 4.54-4.79 (1H, m).

1-(pyrimidin-2-yl)ethanone oxime may be prepared as follows:

Triethylamine (34.2 ml, 245.65 mmol) was added dropwise to a solution of 1-(pyrimidin-2-yl)ethanone (25 g, 204.71 mmol) and hydroxylamine hydrochloride (15.65 g, 225.18 mmol) in ethanol (250 ml) at 20° C., and the reaction heated to 70° C. for 2 hours. The mixture was cooled to room temperature, stirred overnight and evaporated. Water (250 ml) was added and the suspension stirred at room temperature for 3 hours. The product was collected by filtration, washed with water (100 ml), dried on sinter and then under vacuum at 40° C. for 4 days over P2O5 to give 1-(pyrimidin-2-yl)ethanone oxime (20.00 g, 71.2%) as a white solid.

¹H NMR (400.132 MHz, DMSO) δ 2.23 (3H, s), 7.47 (1H, t), 8.84 (2H, d), 11.81 (1H, s)

### Comparative Example A

### (S)-6-Methoxy-2-{2-[3-(pyrimid-2-yl)isoxazol-5-yl]pyrrolidin-1-yl}-4-(5-ethyl-1H-pyrazol-3-ylamino)pyrimidine

(S)-6-Methoxy-2-{2-[3-(pyrimid-2-yl)isoxazol-5-yl]pyrrolidin-1-yl}-4-(5-ethyl-1H-pyrazol-3-ylamino)pyrimidine was prepared by the method described in Example 253 of WO2005/040159.

XRPD analysis indicated the material was amorphous.

### Physical Property Tests and Methods

### LogD

LogD can be measured using the generic shake flask method as described in Lars-Goran Danielsson, Yu Hui Zhang, Trends in Analytical Chemistry, 1996, 15(4), 188-196, and also by the method described in B. Law and D. Temesi, J. Chromatogr. B 748 (2000), 21-30.

Example 1: 3.12

Comparative Example: 3.43

Conclusion: the compound of the present invention has a lower Log D. Reduction in Log D may improve drug properties for example by ameliorating metabolism of the drug.

### Solubility

Solubility values are determined by agitation of compounds in 0.1 M phosphate buffer at pH 7.4 for 24 h at 25° C. The supernatant is separated from undissolved material by double centrifugation and subsequently analyzed and quantified against a standard of known concentration in DMSO using generic HPLC-UV methodology coupled with mass spectral peak identification (J. Med. Chem., 2006, 49(23), 6672-6682).

Example 1: 160 μM

Comparative Example: 9.24 μM

Conclusion: the compound of the present invention has higher solubility. Increased solubility may be advantageous, for example for oral administration, as the rate of adsorption may be increased.
Note: the solubility measured for Example 1 may represent the solubility of amorphous material, measurements carried out on a crystalline sample comprising a mixture of Forms 1 and 3 indicates a solubility of around 18 μM.

### Protein Binding

Protein binding is determined by equilibrium dialysis. A 20 iM concentration of compound is dialyzed against 10% plasma at a temperature of 37° C. for 18 h. The resulting samples are analyzed using generic HPLC-UV methodology coupled with mass spectral peak identification. The reported K1 value is the first apparent association constant [proteináligand]/([protein][ligand]), all concentrations being measured in moles/liter (J. Med. Chem., 2006, 49(23), 6672-6682).

Protein binding can be measured in a high-throughput screen by equilibrium dialysis combined with liquid chromatography and mass spectrometry (Wan, H. and Rehngren, M., J. Chromatogr. A 2006, 1102, 125-134).

Example 1: 5.23% free (rat)

Comparative Example: 2.05% free (rat)

Conclusion: the compound of the present invention shows less protein binding. A reduction in protein binding indicates that there is more free drug (unbound). This may be advantageous as there may be more drug available to act at the target site.

### Biological Assays and Test Methods

### Inhibition of Insulin-Like Growth Factor-1 Receptor Phosphorylation

This immunofluorescence end point cell assay measures the ability of a test compound to reduce the measured levels of IGF1R phosphorylation after IGF1 stimulation in R cells. R⁺ cells are derived by transfection of R⁻ mouse fibroblast cells with human IGF1R. R⁺ cells are routinely cultured in DMEM growth medium (Gibco BRL, 41966) containing 2 mM L-Glutamine (Invitrogen Code no. 25030-024) and 10% (v/v) foetal bovine serum (FBS)) in a 5% CO₂ air incubator at 37° C.

To undertake the assay, the R⁺ cells are seeded at 5×10³ cells/well in DMEM plus 1% foetal calf serum, 1% L-glutamine in 96-well black Packard View plates (PerkinElmer 6005182) and incubated at 37° C. (+5% CO₂) in a humidified incubator. The following day, the plates are dosed with 10 μl of 10× concentrated compound (diluted from 10 mM stock in DMSO and DMEM without serum) and the plates are returned to a humidified 37° C. (+5% CO₂) incubator for 30 minutes. Cells are tested in duplicates in a suitable dose range to accurately measure the compound IC50.

Following the compound treatment, the R⁺ cells are stimulated with a final concentration of 30 nM IGF1 (Gropep IM001) for 20 minutes at 37° C. The IGF1 is dissolved according to the manufacture's instructions to a 26 μM stock solution and diluted in DMEM without serum. Following stimulation the cells are fixed by adding formaldehyde (4% v/v final concentration) and incubating at room temperature for 20 minutes. The fixative solution is removed and the wells are washed twice with 100 μl phosphate buffered saline containing 0.05% Tween20 (PBS-Tween 20) before permeabilising the cells by the addition of 501/well 0.05% Triton in PBS for 10 minutes at room temperature. The permeabilisation solution is removed and the cells washed twice with 100 μl/well PBS-Tween 20 before addition of 50 μl blocking solution containing 2% BSA (Sigma. A-78888)+2% goat serum (DAKO X0907) in PBS. Plates are incubated for 1 hour at room temperature. The blocking solution is aspirated from the wells and 50 μl rabbit dual phospho specific anti-phospho IGF1R/IR (BioSource 44-804) 1/350 diluted in blocking solution is added to the wells. Additionally, in-house antibodies raised against phospho IGF1R were also used at a suitable titre determined for each batch.

Following incubation at room temperature for 1 hour, the antibody solution is removed and the wells washed twice with 100 μl/well PBS-Tween 20. 50 μl/well Alexa Fluor conjugated anti rabbit (Invitrogen/Molecular Probes-A11008) is added to the wells in a dilution of 1/1000 in blocking solution. The plates are incubated at room temperature for one hour. Finally, the plates are washed three times with 100 μl/well PBS-Tween. After addition of 100 μl/well PBS the plates are sealed with a black seal.

The Green Fluorescent phospho IGF1R-associated signal in each well was measured using an Acumen Explorer HTS Reader (TTP Labtech Ltd., Cambridge). Phospho IGF1R-associated fluorescence emission can be detected at 530 nm following excitation at 488 nm. The instrument is a laser-scanning fluorescence microplate cytometer, which samples the well at regular intervals and uses threshold algorithms to identify all fluorescent intensities above the solution background without the need to generate and analyse an image. These fluorescent objects can be quantified and provide a measure of the phospho IGF1R levels in cells. Fluorescence dose response data obtained with each compound was exported into a suitable software package (such as Origin) to perform curve fitting analysis. Phospho-IGF1R levels in response to compound treatment versus stimulated and unstimulated controls were expressed as an IC₅₀ value. This was determined by calculation of the concentration of compound that was required to give a 50% reduction of the maximum phospho—IGF1R signal.

Example 1: 0.00429 (median) (n=11) IC50 (μM)

Comparative Example: 0.00268 (median) (n=7) IC50 (μM)

### Inhibition of Insulin Receptor Phosphorylation

This immunofluorescence end point cell assay measures the ability of a test compound to reduce the measured levels of IR phosphorylation after insulin stimulation in CHOT cells. CHOT cells are Chinese Hamster Ovary cells (CHO) stable transfected with human IR. CHOT cells are routinely cultured in Hams F12 growth medium supplemented with 200 ug/ml Geneticin, 2.5 mM HEPES, 2 mM L-Glutamine (Invitrogen Code no. 25030-024) and 10% (v/v) foetal bovine serum (FBS) in a 5% CO₂ air incubator at 37° C.

To undertake the assay, the CHOT cells are seeded at 5×10³ cells/well in Hams F12 medium plus 2.5 mM HEPES, 1% foetal calf serum and 2 mM L-Glutamine in 96-well black Packard View plates (PerkinElmer 6005182) and incubated at 37° C. (+5% CO₂) in a humidified incubator. The following day, the plates are dosed with 10 μl of 10× concentrated compound (diluted from 10 mM stock in DMSO and Hams F12 without serum) and the plates are returned to a humidified 37° C. (+5% CO₂) incubator for 30 minutes. Cells are tested in duplicates in a suitable dose range to accurately measure the compound IC50.

Following the compound treatment, the CHOT cells are stimulated with a final concentration of 30 nM Insulin (Sigma #I-9278) for 10 minutes at 37° C. The insulin is dissolved according to the manufacture's instructions to a 1.7 mM stock solution and diluted in Hams F12 medium without serum to a 113 nM solution. Following stimulation the cells are fixed by adding formaldehyde (4% v/v final concentration) and incubating at room temperature for 20 minutes. The fixative solution is removed and the wells are washed twice with 100 μl phosphate buffered saline containing 0.05% Tween20 (PBS-Tween 20) before permeabilising the cells by the addition of 50 μl/well 0.05% Triton in PBS for 10 minutes at room temperature. The permeabilisation solution is removed and the cells washed twice with 100 μl/well PBS-Tween 20 before addition of 50 μl blocking solution containing 2% BSA (Sigma. A-78888)+2% goat serum (DAKO X0907) in PBS. Plates are incubated for 1 hour at room temperature. The blocking solution is aspirated from the wells and 50 μl rabbit dual phospho specific anti-phospho IGF1R/IR (BioSource 44-804) 1/350 diluted in blocking solution is added to the wells. Additionally, in-house antibodies raised against phospho IR were also used at a suitable titre determined for each batch.

Following incubation at room temperature for 1 hour, the antibody solution is removed and the wells washed twice with 100 μl/well PBS-Tween 20. 50 μl/well Alexa Fluor conjugated anti rabbit (Invitrogen/Molecular Probes-A11008) is added to the wells in a dilution of 1/1000 in blocking solution. The plates are incubated at room temperature for one hour. Finally, the plates are washed three times with 100 μl/well PBS-Tween. After addition of 100 μl/well PBS the plates are sealed with a black seal.

The Green Fluorescent phospho IR-associated signal in each well was measured using an Acumen Explorer HTS Reader (TTP Labtech Ltd., Cambridge). Phospho IR-associated fluorescence emission can be detected at 530 nm following excitation at 488 nm. The instrument is a laser-scanning fluorescence microplate cytometer, which samples the well at regular intervals and uses threshold algorithms to identify all fluorescent intensities above the solution background without the need to generate and analyse an image. These fluorescent objects can be quantified and provide a measure of the phospho IR levels in cells. Fluorescence dose response data obtained with each compound was exported into a suitable software package (such as Origin) to perform curve fitting analysis. Phospho-IR levels in response to compound treatment versus stimulated and unstimulated controls were expressed as an IC₅₀ value. This was determined by calculation of the concentration of compound that was required to give a 50% reduction of the maximum phospho-IR signal.

Example 1: 0.035 (median) (n=11) IC50 (μM)

Comparative Example: 0.00325 (median) (n=6) IC₅₀ (μM)

Conclusion: whilst the compound of the invention (Example 1) shows comparable activity to a known IGF inhibitor (Comparative Example A) in the inhibition of Insulin-like Growth Factor-1 Receptor Phosphorylation assay, the compound of the invention shows a ten-fold difference in the Inhibition of Insulin Receptor Phosphorylation assay. The selective inhibition of Insulin-like Growth Factor-1 Receptor Phosphorylation over the Inhibition of Insulin Receptor Phosphorylation may be advantageous since such selective compounds may have less effect on insulin signaling, and therefore less disruption of glucose homeostasis and associated toxicological consequences thereof.

### Cytochrome P450 Inhibition Assay

The inhibitory potential (IC₅₀) of test compounds against 5 human cytochrome P450 (CYP) isoforms (1A2, 2C9, 2C19, 3A4 and 2D6) was assessed using an automated fluorescent end point in vitro assay modified from Crespi (Crespi and Stresser, 2000). Microsomal subcellular fractions prepared from Yeast cell lines expressing each human CYP isoform were used as an enzyme source in this assay. The activity of the 5 major human CYPs was determined from the biotransformation of a number of coumarin substrates to fluorescent metabolites, in the presence of NADPH. Inhibition of these CYPs resulted in a decrease in the amount of fluorescent metabolite formed. Comparison of the fluorescence observed in the presence of varying concentrations of test compound with that seen in its absence allowed an IC₅₀ value to be calculated. Initial experiments were performed to optimise the kinetic parameters of the assay and these have been listed in Table 1. Stock solutions of each CYP, with its respective substrate, were prepared in phosphate buffer pH7.4 (see Table 1) and 178 μl was added to the well of a black solid, flat bottom, 300 μl 96 well is microtitre plate (Corning Costar). Test compounds were serially diluted in DMSO/acetonitrile and added (2 μl) to the reaction to give final concentrations of 0.1, 0.3, 1, 3 and 10 μM. After pre-incubating at 37° C. for 5 min the reactions were started with addition of NADPH (20 μl, concentration shown in Table 1). The final solvent content in each incubation was <=2% (1% from the test compound and a maximum of 1% from the substrate). The appropriate solvent controls and substrate blanks were included in each experiment to assess control activity and identify any inherent fluorescence due to the test compounds. In addition, known inhibitors of each CYP were included as positive controls (see Table 3 for inhibitor concentrations and expected IC₅₀ ranges). The reactions were stopped at defined timepoints (see Table 1) by quenching with 100 μl of solvent (acetonitrile:0.5M Tris buffer 80:20 v/v). The plates were read on a fluorimeter (Spectrafluor Plus) at the appropriate excitation and emission wavelengths (listed in Table 2) and the percent activity, corrected for control, was plotted against the test compound concentration. The IC₅₀ (the concentration of test compound required to cause 50% inhibition of metabolic activity) for each CYP was then determined from the slope of these plots.

TABLE 1
Concentrations of assay reagents and assay conditions.

| | CYP solution | | | Phosphate | | Incubation |
|---|---|---|---|---|---|---|
| | (pmol/ | | Substrate | Buffer | NADPH | time |
| CYP | 200 μl) | Substrate | (uM) | (M) | (μM) | (min) |
| 1A2 | 1 | 3-cyano-7- | 3 | 0.1 | 250 | 20 |
| | | ethoxy-coumarin | | | | |
| | | (CEC) | | | | |
| 2C9 | 3 | 7-methoxy-4- | 50 | 0.025 | 250 | 40 |
| | | trifluoromethyl- | | | | |
| | | coumarin (MFC) | | | | |
| 2C19 | 5 | 7-methoxy-4- | 50 | 0.05 | 250 | 60 |
| | | trifluoromethyl- | | | | |
| | | coumarin (MFC) | | | | |
| 2D6 | 3 | 7-methoxy-4- | 20 | 0.1 | 60 | 35 |
| | | (aminomethyl)- | | | | |
| | | coumarin | | | | |
| | | (MAMC) | | | | |
| 3A4 | 5 | 7-benzyloxy-4- | 15 | 0.1 | 250 | 35 |
| | | (trifluoromethyl)- | | | | |
| | | coumarin (BFC) | | | | |

TABLE 2
Excitation and emission wavelengths used by Spectrafluor
Plus Fluorimeter to detect fluorometric metabolites. CEC
and HFC were obtained from Ultrafine Chemicals; CHC
was obtained from Molecular Probes; MFC, MAMC, HAMC
and BFC were obtained from Gentest Corporation.

| | | | Excitation | Emission |
|---|---|---|---|---|
| CYP | Substrate | Metabolite | λ (nm) | λ (nm) |
| 1A2 | 3-cyano-7-ethoxy- | 3-cyano-7-hydroxy- | 405 | 460 |
| | coumarin (CEC) | coumarin (CHC) | | |
| 2C9 | 7-methoxy-4- | 7-hydroxy-4- | 405 | 535 |
| | trifluoromethyl- | trifluoromethyl- | | |
| | coumarin (MFC) | coumarin (HFC) | | |
| 2C19 | 7-methoxy-4- | 7-hydroxy-4- | 405 | 535 |
| | trifluoromethyl- | trifluoromethyl- | | |
| | coumarin (MFC) | coumarin (HFC) | | |
| 2D6 | 7-methoxy-4- | 7-hydroxy-4- | 390 | 460 |
| | (aminomethyl)- | (aminomethyl)- | | |
| | coumarin (MAMC) | coumarin (HAMC) | | |
| 3A4 | 7-benzyloxy-4- | 7-hydroxy-4- | 405 | 535 |
| | (trifluoromethyl)- | trifluoromethyl- | | |
| | coumarin (BFC) | coumarin (HFC) | | |

TABLE 3
Known inhibitors and optimised experimental conditions for each
of the 5 human CYP isoforms. Fluvoxamine was obtained from Tocris
Cookson Ltd; Sulphaphenazole and Quinidine were obtained from
Sigma; Omeprazole was obtained from AstraZeneca;
Ketoconazole was obtained from Ultrafine Chemicals.

| | | Range of | |
|---|---|---|---|
| | | standard inhibitor | |
| | Substrate | concentrations | IC₅₀ range |
| CYP | (μM) | (μM) | (μM) |
| 1A2 | 3 | Fluvoxamine | 0.01-0.07 |
| | | 1, 0.3, 0.1, 0.03, 0.01 | |
| 2C9 | 50 | Sulphaphenazole | 0.1-1.0 |
| | | 10, 3, 1, 0.3, 0.1 | |
| 2C19 | 50 | Omeprazole | 1.5-4.6 |
| | | 10, 3, 1, 0.3, 0.1 | |
| 2D6 | 20 | Quinidine | 0.003-0.03 |
| | | 0.1, 0.03, 0.01, 0.003, | |
| | | 0.001 | |
| 3A4 | 15 | Ketoconazole | 0.005-0.015 |
| | | 0.25, 0.075, 0.025, | |
| | | 0.0075, 0.0025 | |

### Comparative Testing of Example 1 and Comparative Example A

| | Ic50 | Ic50 | Ic50 | Ic50 | Ic50 |
|---|---|---|---|---|---|
| | 1A2 | 2C9 | 2C19 | 2D6 | 3A4 |
| Example 1 | >10 | >10 | >10 | >10 | >10 |
| Comparative | 1.87 | >10 | >10 | >10 | >10 |
| Example A | | | | | |

Conclusion: Compounds of the present invention (Example 1) while showing good IGF inhibition, also show decreased Cytochrome P450 inhibition when compared to a known IGF inhibitor (Comparative Example A). Low inhibition of Cytochrome P450 is desirable to ameliorate potential drug: drug interactions.
hERG
hERG can be tested according to the methods described in Journal of Pharmacolgical and Toxicological Methods 2006, 54, 189-199.

Example 1: >32 (IC₅₀)

Comparative Example: 25.2 (IC₅₀)

Conclusion: Inhibition of the hERG (human ether-a-go-go-related gene) ion channel is a major cause of changes in cardiac rhythm (changes in ECG) and more specifically increases in the QT interval. Large changes to the QT interval can result in arrhythmias and sudden death. hERG activity is a predictor of QT interval which is a surrogate marker for risk of severe cardiac arrhythmia and sudden death. The compound of the present invention has a higher IC₅₀ value (is a less effective inhibitor). Reduced activity against the hERG channel is an advantageous property as it eliminates or minimises this risk of serious adverse effect.

1. A compound of formula (I): 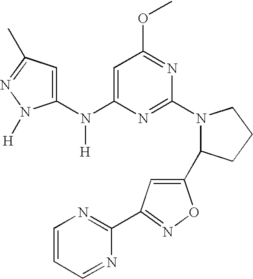
or a pharmaceutically acceptable salt thereof.

2. A compound according to claim 1 wherein the compound is a compound of formula (Ia): 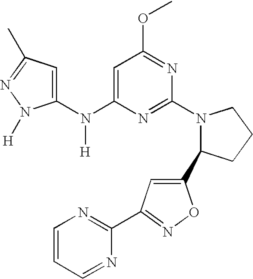
or a pharmaceutically acceptable salt thereof.

3. A compound according to claim 2 which is:
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 2, which has an X-ray powder diffraction pattern with specific peaks at 2θ=6.906, 9.061, 10.693, 12.256, 14.393, 15.067, 15.903, 17.003, 18.317, 19.823, 21.458, 21.74, 20.603, 23.214, 24.635 and 25.061° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 1, which has an X-ray powder diffraction pattern with specific peaks at 2θ=5.476, 7.671, 7.95, 10.749, 14.513, 15.172, 15.584, 18.507, 20.226, 20.983, 22.068, 23.251, 23.567, 24.607, 26.189, 26.796 and 27.512° when measured using CuKa radiation;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 3, which has an X-ray powder diffraction pattern with specific peaks at 2θ=5.605, 6.808, 10.149, 13.221, 16.193, 18.506, 18.848, 19.814, 20.389, 20.827, 22.066, 23.159, 23.94, 24.288, 25.006, 26.54 and 26.924° when measured using CuKa radiation; or
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 4, which has an X-ray powder diffraction pattern with specific peaks at 2θ=7.304, 7.56, 10.365, 15.766, 16.027, 17.982, 18.74, 20.172, 20.46, 21.234, 23, 23.249, 24.391, 25.516, 26.772 and 27.297° when measured using CuKa radiation.

4. A compound according to claim 2 which is:
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 2, having an X-ray powder diffraction pattern substantially the same as the X-ray powder diffraction pattern shown in Figure A;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 1, having an X-ray powder diffraction pattern substantially the same as the X-ray powder diffraction pattern shown in Figure B;
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 3, having an X-ray powder diffraction pattern substantially the same as the X-ray powder diffraction pattern shown in Figure C; or
(S)-6-Methoxy-2-{2-[3-(pyrimid-2-yl)isoxazol-5-yl]pyrrolidin-1-yl}-4-(5-methyl-1H-pyrazol-3-ylamino)pyrimidine, Form 4, having an X-ray powder diffraction pattern substantially the same as the X-ray powder diffraction pattern shown in Figure D.

5. A compound of formula (I) or (Ia), or a pharmaceutically acceptable salt thereof, as claimed in claims 1 or 2 for use in therapy of the human or animal body.

6. The use of a compound of formula (I) or (Ia), or a pharmaceutically acceptable salt thereof, as claimed in claims 1 or 2 in modulating insulin-like growth factor-1 receptor (IGF-1R) activity in a human or animal.

7. A method of treating cancer which comprises administering to a patient in need thereof a therapeutically effective amount of a compound of formula (I), or a pharmaceutically acceptable salt thereof, as claimed in claims 1 or 2.

8. A method of modulating insulin-like growth factor-1 receptor (IGF-1R) activity which comprises administering to a patient in need thereof a therapeutically effective amount of a compound of formula (I) or (Ia), or a pharmaceutically acceptable salt thereof, as claimed in claims 1 or 2.

9. A pharmaceutical composition comprising a compound of formula (I) or (Ia), or a pharmaceutically acceptable salt thereof, as claimed in claims 1 or 2, in association with a pharmaceutically acceptable adjuvant, diluent or carrier.

10. A process for the preparation of a pharmaceutical composition of the invention which comprises mixing a compound of formula (I) or (Ia), or a pharmaceutically acceptable salt thereof, as claimed in claims 1 or 2, with a pharmaceutically acceptable adjuvant, diluent or carrier.
